(19) United States
(12) Patent Application Publication
(10) Pub. No.: US 2014/0012141 A1
(43) Pub. Date: Jan. 9, 2014

(54) OPTICAL TOMOGRAPHIC IMAGING OTOSCOPE WITH INTEGRATED DISPLAY AND DIAGNOSIS

(71) Applicant: Kyungpook National University Industry-Academic Cooperation Foundation, Daegu (KR)

(72) Inventors: Jee Hyun Kim, Daegu (KR); Nam Hyun Cho, Daegu (KR); Hyeong Il Kwon, Daegu (KR); Un Sang Jung, Daegu (KR); Hyo Sang Jeong, Daegu (KR)

(73) Assignee: Kyungpook National University Industry-Academic Cooperation Foundation, Daegu (KR)

(21) Appl. No.: 14/021,451

(22) Filed: Sep. 9, 2013

Related U.S. Application Data
(63) Continuation of application No. PCT/KR2011/006599, filed on Sep. 7, 2011.

(30) Foreign Application Priority Data
Mar. 8, 2011 (KR) 10-2011-0020524

(51) Int. Cl. A61B 1/227

(52) U.S. Cl. CPC A61B 1/227
USPC 600/476

(57) ABSTRACT

A diagnosis-and-display integrated optical tomographic imaging otoscope for examining otitis media. A hollow casing includes an ear specular disposed on a front surface, a display including an LCD disposed on a rear surface, and a manipulating handle on a lower part. An image-photographing part includes a CCD camera inside the casing, and photographs an ear drum image of a patient through the ear specular. A section-photographing part includes a collimator and a galvanometer mirror inside the casing, and photographs section images of the ear drum and a middle ear of the patient. The ear drum image obtained by the image-photographing part and the section images of the ear drum and the middle ear are obtained in a non-incision method by the section-photographing part, and are displayed in real time on the LCD such that desirable images can be stored.

### CROSS-REFERENCE TO RELATED APPLICATIONS

The present application is a continuation of International Application No. PCT/KR2011/006599 filed Sep. 7, 2011, which claims priority to Korean Application No. 10-2011-0020524 filed Mar. 8, 2011, which applications are incorporated herein by reference.

### TECHNICAL FIELD

The present invention relates to an apparatus for examining an otitis media, and more particularly, to a diagnosis-and-display integrated optical tomographic imaging otoscope which can photograph the surface image of the ear drum, the section of the ear drum and condition of the middle ear in a non-incision method and display the photographed images on a liquid crystal display (LCD) of a display. The state of a patient can be promptly examined, thereby significantly increasing the convenience of a diagnosis, diagnosing procedures can be simplified and causes for a disease can be diagnosed at an early stage so that prescriptions according to the causes can be suitably prepared.

### BACKGROUND ART

In general, otitis media refers to inflammation in the middle ear, the space positioned inside the eardrum. In particular, this is a prevalent disease in children who visit hospitals. Otorhinolaryngological apparatuses that examine only the condition of the eardrum have limited ability to diagnose otitis media.

Methods of diagnosing otitis media of the related art examined only the condition of the surface of the eardrum based on clinical symptoms and using an otoscope. However, according to these methods, it is difficult to diagnose causes for the defection of the otitis media at an early stage, differentiate between bacterial and general infections, and examine the condition of the middle ear positioned below the ear drum. Accordingly, there are many obstacles to address correct diagnosis.

In order to take an image of the section of the ear drum of a patient having symptoms of the otitis media, a tomography apparatus such as a computerized tomography (CT) apparatus or a magnetic resonance imaging (MRI) apparatus. However, it is much difficult to accurately diagnose patients since the resolutions of such apparatuses are limited.

In addition, these methods also act as factors that complicate diagnostic procedures and make it inconvenient in the diagnosis of patients. Early diagnosis of a disease is difficult. It is difficult to examine a patient since a separate monitor device is used. Furthermore, it is difficult to obtain diagnosis data for a patient since it is not easy to store an image.

For example, the types of otoscope of the related art can be basically divided into two types. Specifically, the types of otoscope can be divided into a lens type otoscope 10 shown in FIG. 1a with which an examiner by approaching his/her eyes to a lens and an image otoscope 20 as shown in FIG. 1b which includes an otoscope 22 and a camera 24 which is connected to the otoscope to take an image.

The related-art lens type otoscope 10 has a drawback in that it is difficult for a doctor to visually examine the ear drum. It is difficult to correctly examine the condition of the ear drum since the ear drum is small and dark in most cases. In order to examine the condition of the ear drum, a diagnosis must be performed by replacing a magnification lens disposed inside the otoscope depending on the eyesight of the doctor.

This lens type otoscope 10 has the danger of an erroneous diagnosis since the doctor must make subjective diagnosis by examining only the surface condition of the ear drum, and acts as a factor that can delay the time for diagnosis.

In addition, the related-art image otoscope 20 using the camera can obtain a clear image by radiating light onto the ear drum. However, since light is radiated through an optical fiber and the image is displayed on a large monitor, the cost of this apparatus increases.

In addition, there are disadvantages in that the resolution of the camera is not satisfactory, colors are not clearly distinguished, and an image that is obtained at the same time as a patient is being diagnose cannot be examined.

In addition, alternatively, an endoscope type otoscope was proposed, as shown in FIG. 2a. This related-art endoscope type otoscope 30 is of the type that is most widely used in the otorhinolaryngology. Since a high-resolution camera is used which has bright light, the condition of the ear drum can be more easily examined than by the lens type otoscope 10 or the imaging otoscope 20.

However, this related-art endoscope type otoscope 30 has a drawback in that it diagnoses only the surface condition of the ear drum, as in existing otoscopes. It is therefore impossible to examine the condition of the middle ear behind the ear drum.

In addition, this apparatus is required to illuminate dark regions using an expensive optical fiber, which becomes a factor that increases the unit price of the apparatus. There are other drawbacks in that a separate monitor is required, it is difficult to examine the image of the ear drum, and the size of the apparatus is increased.

Alternatively, a surgical microscope 40 shown in FIG. 2b was proposed. The related-art surgical microscope 40 can also diagnose only the surface of the ear drum like the lens type otoscope 10, the imaging otoscope 20, or the endoscope type otoscope 30. In addition, the size of the apparatus is very large, the price of the apparatus is expensive, and the lens of the apparatus must be adjusted depending on the eyesight of the user, which is inconvenient.

### SUMMARY

The present invention has been made to solve the foregoing problems with the related art, and therefore the present invention is intended to provide a diagnosis-and-display integrated optical tomographic imaging otoscope that can photograph both the condition of the section of the ear drum and the condition of the middle ear together with the surface image of the ear drum that is to be diagnosed, display the photographed images on a display unit of the otoscope, and promptly store desirable images so as to be used.

The present invention is also intended to provide a diagnosis-and-display integrated optical tomographic imaging otoscope that can examine not only the surface condition of the ear drum but also the condition of the section of the ear drum and a disease in the middle ear positioned below the ear drum using an obtained two-dimensional (2D) image, thereby reducing the chance of an erroneous diagnosis than a related-art otoscope, examine points in the depth direction into the middle ear so that the thickness of the ear drum can be measured in real time and a diseased region can be accurately determined in real time, thereby obtaining the efficiency and reliability of the diagnosis, and simplify procedures of diagnosis, thereby realizing the convenience of diagnosis and easily obtaining data.

In order to realize the foregoing objects, in an aspect of the present invention, provided is a diagnosis-and-display integrated optical tomographic imaging otoscope for examining otitis media that includes: a casing having a hollow structure of a predetermined size, wherein the casing includes an ear specular disposed on a front surface, a display including a liquid crystal display (LCD) disposed on a rear surface, and a manipulating handle on a lower part; an image-photographing part including a charge-coupled device (CCD) camera inside the casing, wherein the image-photographing part photographs an ear drum image of a patient through the ear specular; and a section-photographing part including a collimator and a galvanometer mirror inside the casing, wherein the section-photographing part photographs section images of the ear drum and a middle ear of the patient. The ear drum image obtained by the image-photographing part and the section images of the ear drum and the middle ear obtained by the section-photographing part are displayed in real time on the LCD such that desirable images can be stored.

It is preferred that the casing may include an illumination visible light source which is disposed on the ear specular to illuminate an inside of an ear of the patient.

It is preferred that the image-photographing part may include magnification lenses downstream of the CCD camera, a color correction filter mirror (dichroic mirror) downstream of the magnification lenses, and a focus lens downstream of the color correction filter mirror, whereby the CCD camera photographs the ear drum image of the patient in real time through the ear specular.

It is preferred that the section-photographing part may include a galvanometer mirror downstream of the collimator, a color correction filter mirror downstream of the galvanometer mirror, and a focus lens downstream of the color correction filter mirror, whereby the section images of the ear drum and the middle ear of the patient are photographed in real time through the ear specular.

It is preferred that the color correction filter mirror may be disposed at an inclined position, and direct light from the focus lens to the CCD camera of the image-photographing part by allowing the light to pass through and light from the collimator of the section-photographing part to the focus lens by reflecting the light.

It is preferred that the display part concurrently may display the ear drum image obtained from the image-photographing part on one portion of the LCD and the section images of the ear drum and the middle ear obtained from the section-photographing part on the other portion of the LCD.

It is preferred that the handle may have a button on a portion thereof. When the button is pressed, the ear drum image obtained from the image-photographing part and the section images of the ear drum and the middle ear obtained from the section-photographing part are stored in a memory which is disposed inside a control part.

According to the diagnosis-and-display integrated optical tomographic imaging otoscope according to the present invention, the image-photographing part can photograph an ear drum image, and the section-photographing part can photograph section images of the ear drum and the middle ear, such that the images can be concurrently displayed on the LCD of the display disposed on the rear surface of the casing. Desirable images can be stored for use in the memory inside the control part.

According to the present invention, it is possible to concurrently examine the condition of the section of the ear drum and the condition of the middle ear, provide better resolution than the existing CT or MRI apparatus, simplify the disease-diagnosing procedures, diagnose causes for the defection of the otitis media at an early stage so that prescriptions according to the causes can be suitably prepared, and reliably differentiate between bacterial and general infections so that errors in related diagnoses can be reduced.

Furthermore, according to the invention, not only the surface condition of the ear drum but also the condition of the section of the ear drum and any disease of the middle ear positioned below the ear drum can be concurrently examined based on the 2D images obtained from the image-photographing part and the section-photographing part, thereby reducing the chance of an erroneous diagnosis. It is possible to examine points in the depth direction into the middle ear so that the thickness of the ear drum can be measured in real time and a diseased region can be accurately determined in real time, thereby obtaining the efficiency and reliability of the diagnosis, and simplify procedures of diagnosis, thereby realizing the convenience of diagnoses and easily obtaining data.

### BRIEF DESCRIPTION OF DRAWINGS

FIG. 1a is a perspective view showing the external structure of a lens type otoscope of the related art;

FIG. 1b is a perspective view showing the external structure of an imaging otoscope of the related art;

FIG. 2a is a perspective view showing the structure of an endoscope type otoscope of the related art;

FIG. 2b is a perspective view showing the structure of a surgical microscope of the related art;

FIG. 3 is a view showing the external structure of a diagnosis-and-display integrated optical tomographic imaging otoscope with according to the present invention;

FIG. 4 is an exploded view of the diagnosis-and-display integrated optical tomographic imaging otoscope according to the present invention;

FIG. 5 is a side view showing the internal structure of the diagnosis-and-display integrated optical tomographic imaging otoscope according to the present invention; and

FIG. 6 is an operational explanatory view illustrating the operating principle of the diagnosis-and-display integrated optical tomographic imaging otoscope according to the present invention;

FIG. 7 is an explanatory view illustrating the condition in which an image of the ear drum and an image of the section of the middle ear are displayed on a display provided at the rear surface of the casing of the diagnosis-and-display integrated optical tomographic imaging otoscope according to the present invention;

FIG. 8a is a picture showing an ear drum image obtained from the diagnosis-and-display integrated optical tomographic imaging otoscope according to the present invention;

FIG. 8b is a picture showing section images of the ear drum and the middle ear obtained from the diagnosis-and-display integrated optical tomographic imaging otoscope according to the present invention; and

FIG. 8c is a graph showing a specific portion obtained by graphing the section images of the ear drum and the middle ear obtained from the diagnosis-and-display integrated optical tomographic imaging otoscope according to the present invention (A-Scan), in which the thickness of the ear drum and the condition of the middle ear are examined.

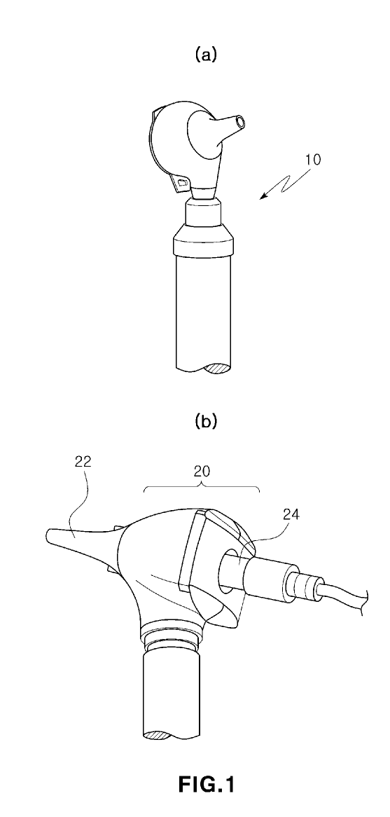

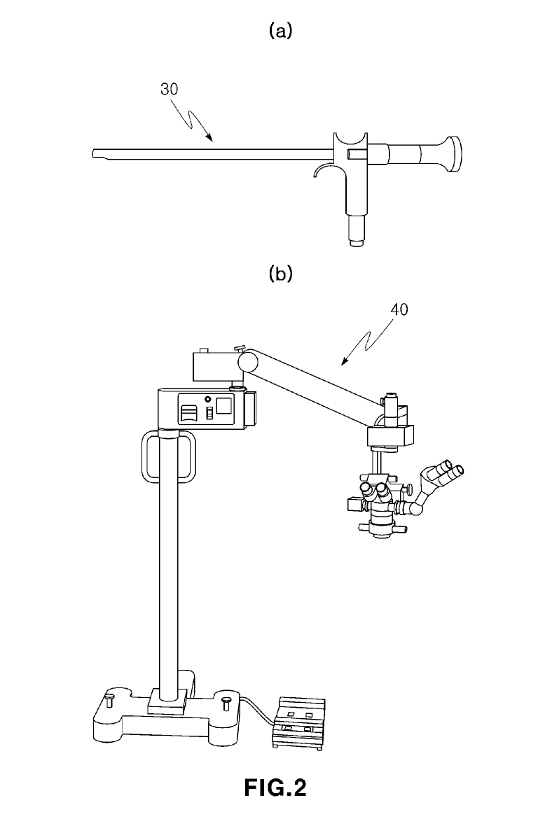

### DETAILED DESCRIPTION

Reference will now be made in detail to various embodiments of the present invention, examples of which are illustrated in the accompanying drawings and described below.

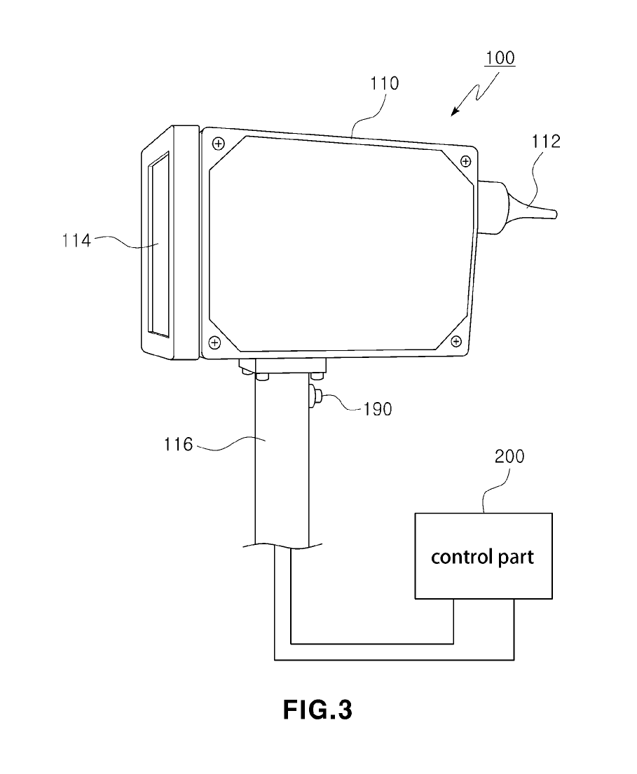

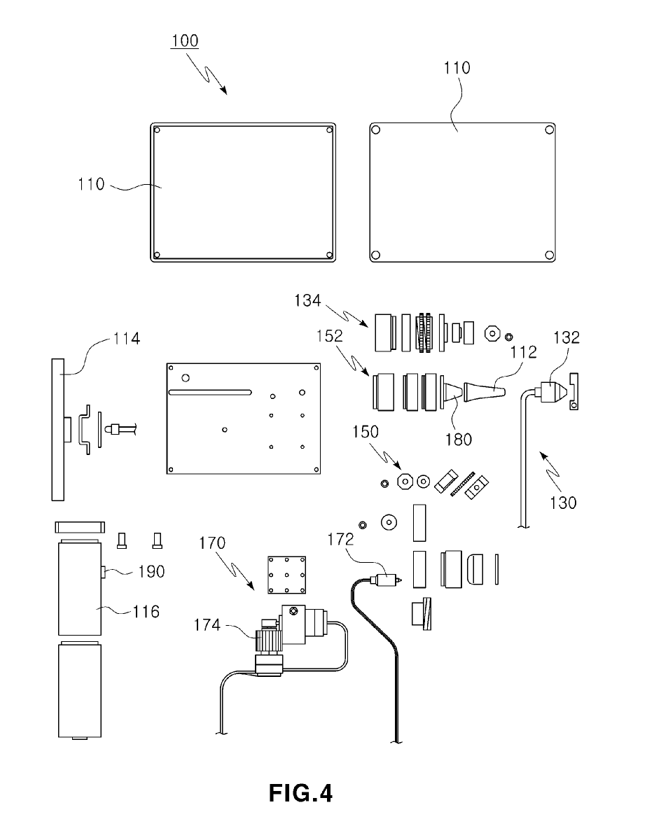

A diagnosis-and-display integrated optical tomographic imaging otoscope 100 according to the present invention is an apparatus for examining otitis media, and as shown in FIG. 3 to FIG. 6, includes a hollow casing 110 having a certain size.

The casing 110 is configured, preferably, as a rectangular box. An ear specular 112 is disposed on the front side, a display 114 having a liquid crystal display (LCD) is disposed on the rear side, and a manipulating handle 116 is disposed on the underside.

In addition, the diagnosis-and-display integrated optical tomographic imaging otoscope 100 according to the present invention also includes a image-photographing part which has a charge-coupled device (CCD) camera 132 inside the casing 110, and takes an image of the ear drum of a patient through the ear specular 112.

This image-photographing part has a magnification lenses 134 downstream of the CCD camera 132 and a color correction filter mirror (dichroic mirror) 150 downstream of the lenses 134.

A focus lens 152 is also provided downstream of the color correction filter mirror 132, such that the CCD camera 132 takes an image of the ear drum of a patient in real time through the ear specular 112.

In this image-photographing part, the CCD camera 132 photographs an image of the surface of the ear drum “P” inside the ear through the magnification lens 134, through the color correction filter mirror 150 and the focus lens 152, and through the ear specular 112 in order to take an image of the ear drum “P,” a small object inside the ear.

In addition, an obtained ear drum image S1 appears on an LCD screen of the display 114 which is disposed on the rear side of the casing 110 through a control part 200 which will be described later.

In addition, the diagnosis-and-display integrated optical tomographic imaging otoscope 100 according to the present invention includes a collimator 172 and a galvanometer mirror 174 inside the casing 110. The diagnosis-and-display integrated optical tomographic imaging otoscope 100 also includes a section-photographing part 170 which photographs the sections of the ear drum “P” and the middle ear “T” of the patient through the ear specular 112.

This section-photographing part 170 has the galvanometer mirror 174 downstream of a collimator 172, the color correction filter mirror 150 is provided downstream of the galvanometer mirror 174, and the focus lens 152 is provided downstream of the galvanometer mirror 174. With this configuration, the section-photographing part 170 photographs the sections of the ear drum “P” and the middle ear “T” through the ear specular 112.

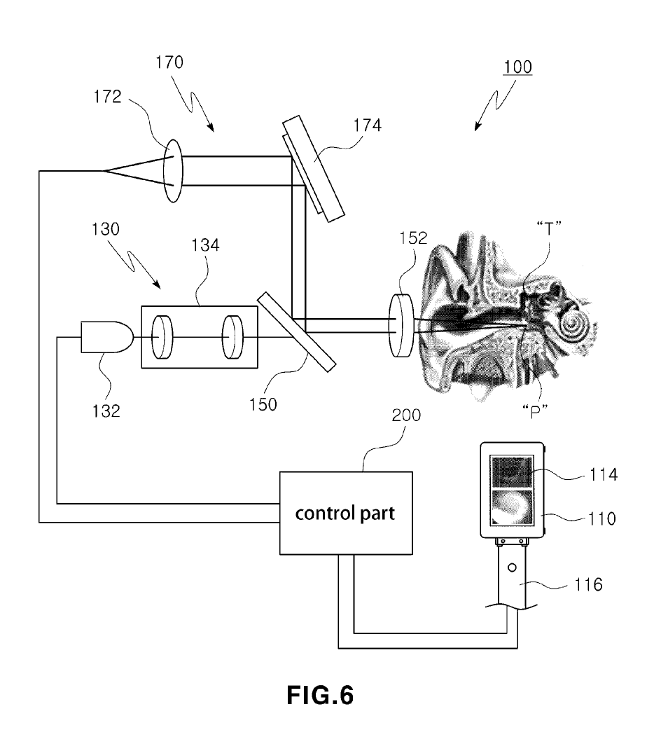

As shown in FIG. 6, the section-photographing part 170 photographs the sections of the ear drum “P” and the middle ear “T” by forming a focus at the tip of ear specular 112 through the collimator 172 and the galvanometer mirror 174 and through the color correction filter mirror 150 and the focus lens 152.

The obtained images S2 of the sections (section images S2) of the ear drum “P” and the middle ear “T” are displayed concurrently with the ear drum image S1 on the LCD screen of the display 114 disposed on the rear side of the casing 110 by the control part 200.

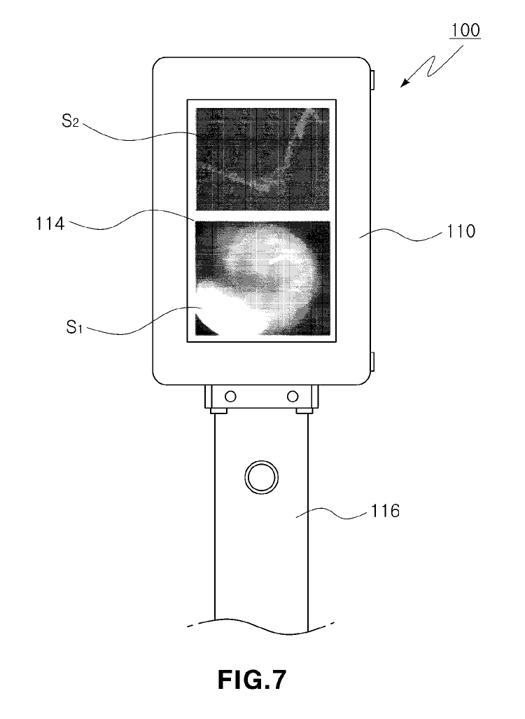

As for the ear drum image S1 obtained as above and the section images S2 of the ear drum “P” and the middle ear “T,” the ear drum image S1 obtained by image-photographing part can be displayed on one portion, i.e. the lower portion, of the LCD, and the section images S2 of the ear drum “P” and the middle ear “T” obtained by the section-photographing part 170 can be displayed on the other portion, i.e. the upper portion, of the LCD, as shown in FIG. 7.

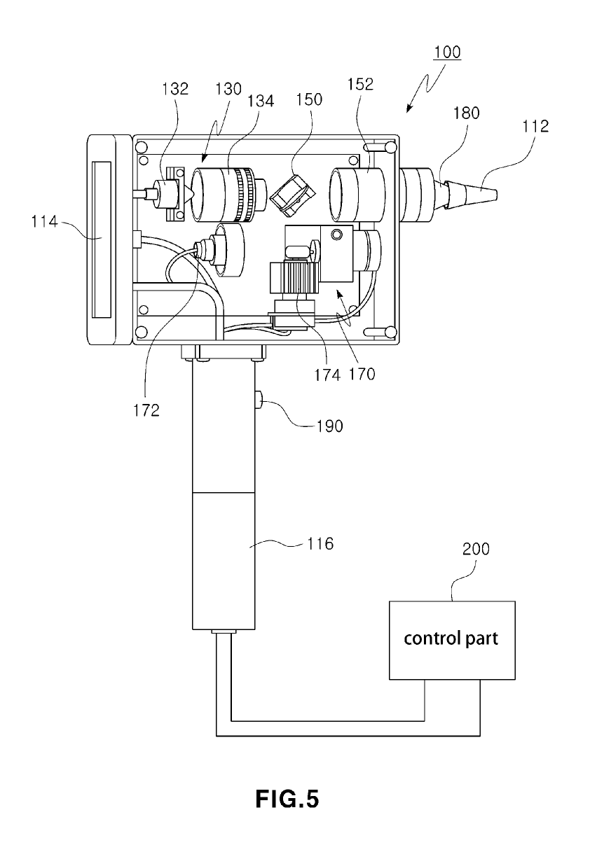

In the diagnosis-and-display integrated optical tomographic imaging otoscope 100 according to the present invention as described above, the color correction filter mirror 150 is commonly used by the image-photographing part and the section-photographing part 170. As shown in FIG. 5 and FIG. 6, the color correction filter mirror 150 is disposed at the inclined position. The color correction filter mirror 150 directs light from the focus lens 152 to the CCD camera 132 of the image-photographing part by allowing the light to pass through and light from the focus lens 152 to the galvanometer mirror 174 and the collimator 172 of the section-photographing part 170 by reflecting the light.

This color correction filter mirror 150 is a mirror acting as a filter that reflects a certain wavelength range of visible light while allowing the remaining wavelength range of visible light to pass through. Unlike filters having different uses, the color correction filter mirror 150 is characterized by reflecting the complementary color of the color that it transmits.

The color correction filter mirror 150 can be used, for example, in a color magnifier in order to control the impression of a color from a color printer, and can be implemented as any structure that is typically used in the art.

In addition, the diagnosis-and-display integrated optical tomographic imaging otoscope 100 according to the present invention is configured such that an illumination visible light source 180 is disposed at the ear specular 112 disposed at the front surface of the casing 110 in order to illuminate the inside of the ear of the patient. The visible light source 180 can be configured such that light is provided through an optical fiber from an external light source (not shown).

Furthermore, as shown in FIG. 3 to FIG. 3, the diagnosis-and-display integrated optical tomographic imaging otoscope 100 according to the present invention includes a button 190 at one portion of the handle 116. With this configuration, when the button 190 is pressed, the ear drum image S1 obtained from the image-photographing part and the section images S2 of the ear drum “P” and the middle ear “T” obtained from the section-photographing part 170 are stored in a memory (not shown) which is disposed inside the control part 200.

The control part 200 is preferably implemented as a small computer or microcomputer having a typical structure, in which the memory which stores various pieces of information therein and a calculator for processing information are provided.

The diagnosis-and-display integrated optical tomographic imaging otoscope 100 according to the present invention displays the ear drum image S1 obtained from the image-photographing part and the section images S2 of the ear drum “P” and the middle ear “T” obtained from the section-photographing part 170 in real time on the LCD of the display 114, and stores desirable images.

Specifically, a doctor can examine the surface of the ear drum of a patient and concurrently photograph the surface of the ear drum “P” and the sections of the ear drum “P” and the middle ear “T” of the patient in a non-incision method. The photographed images S1 and S2 are displayed on the LCD screen of the display 114 which is disposed on the rear surface of the casing 110 such that the images can be examined in real time. It is possible to easily store desirable images using the button 190 provided on the handle 116. It is possible to examine a disease and measure the thickness of the ear drum depending on positions in the depth direction from the outer ear into the middle ear based on the 2D images obtained in this fashion. Consequently, it is easy to realize the efficiency and reliability of a diagnosis.

For this, in the diagnosis-and-display integrated optical tomographic imaging otoscope 100 according to the present invention, as shown in FIG. 6, the CCD camera 132 of the image-photographing part takes the surface image of the ear drum “P” through the magnification lens 134, the color correction filter mirror 150 and the focus lens 152.

As shown in FIG. 7, the control part 200 controls the obtained image S1 of the ear drum to appear on the lower portion of the LCD screen of the display 114 disposed on the rear side of the casing 110.

In addition, in the diagnosis-and-display integrated optical tomographic imaging otoscope 100 according to the present invention, the section-photographing part 170 photographs the sections of the ear drum “P” and the middle ear “T” of a patient through the collimator 172, the galvanometer mirror 174, the color correction filter mirror 150 and the focus lens 152.

In addition, the control part 200 controls the section images S2 of the ear drum P and the middle ear T photographed as above and the ear drum image S1 to concurrently appear side by side on the upper portion of the LCD screen of the display 114 which is disposed on the rear side of the casing 110.

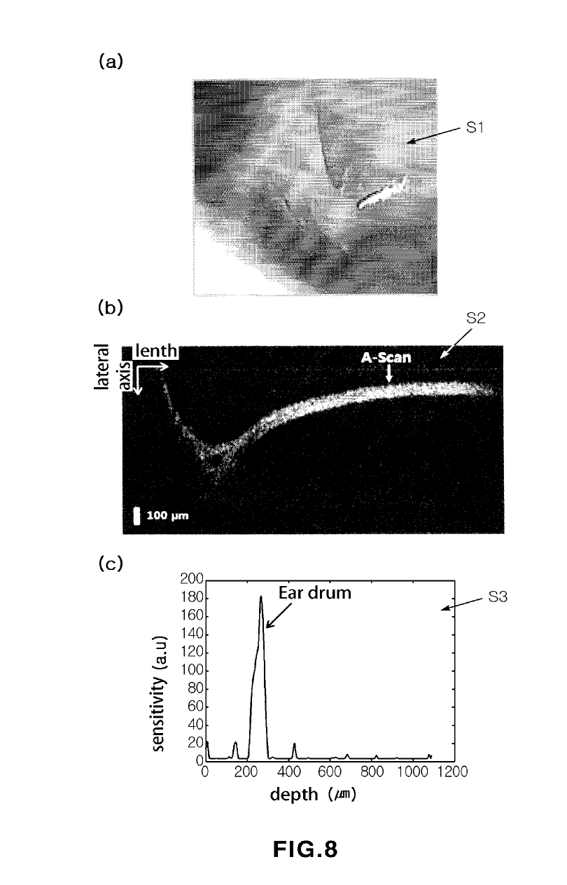

FIG. 8a shows an ear drum image S1 of a normal person actually photographed using the diagnosis-and-display integrated optical tomographic imaging otoscope 100 according to the present invention, i.e the image of the entire ear drum obtained through the CCD camera 132 of the image-photographing part, and FIG. 8b is section images S2 of the ear drum obtained through the section-photographing part 170.

In the section image S2 of FIG. 8b, the width indicates the horizontal direction of the ear drum, and the height indicates the depth direction toward the middle ear. FIG. 8c is an image graph S3 obtained by graphing a specific portion of the images of FIG. 8b (A-Scan), in which the thickness of the ear drum and the condition of the middle ear are examined. In the image graph S3, the width is the depth direction toward the middle ear, and the height is the strength depending on the depth.

In the diagnosis-and-display integrated optical tomographic imaging otoscope 100 according to the present invention as described above, the image-photographing part photographs the ear drum image S1, and the section-photographing part 170 photographs the section images S2 of the ear drum P and the middle ear T. The photograph images are concurrently displayed on the LCD of the display 114 disposed on the rear surface of the casing 110, such that desirable images S1 and S2 can be stored in the memory inside the control part 200 for use.

Accordingly, in the diagnosis-and-display integrated optical tomographic imaging otoscope 100 according to the present invention, it is possible to concurrently examine the condition of the section of the ear drum “P” and the condition of the middle ear “T,” provide better resolution than the existing CT or MRI apparatus, simplify the disease-diagnosing procedures, diagnose causes for the defection of the otitis media at an early stage so that prescriptions according to the causes can be suitably prepared, and reliably differentiate between bacterial and general infections so that errors in related diagnoses can be reduced.

Furthermore, in the diagnosis-and-display integrated optical tomographic imaging otoscope 100 according to the present invention, not only the surface condition of the ear drum “P” but also the condition of the section of the ear drum “P” and any disease of the middle ear “T” positioned below the ear drum “P” can be concurrently examined based on the 2D images S1 and S2 obtained from the image-photographing part and the section-photographing part 170, thereby reducing the chance of an erroneous diagnosis. It is possible to examine points in the depth direction into the middle ear so that the thickness of the ear drum can be measured in real time and a diseased region can be accurately determined in real time, thereby obtaining the efficiency and reliability of the diagnosis, and simplify procedures of diagnosis, thereby realizing the convenience of diagnoses and easily obtaining data.

Although the present invention has been described hereinabove with respect to the exemplary embodiments in conjunction with the drawings, it should be understood that the present invention is not limited to the foregoing configurations. A person having ordinary skill in the art can alter or modify the present invention in various forms without departing from the technical idea or the scope of right of the present invention defined in the appended Claims. For instance, although the ear drum image S1 and the section images S2 of the ear drum “P” and the middle ear “T” can be concurrently displayed on the LCD of the display 114, it is of course possible to alternately and sequentially display the images by manipulating the button 190. It should be understood, however, all such alterations and modifications fall within the scope of the present invention.

1. A diagnosis-and-display integrated optical tomographic imaging otoscope for examining otitis media, comprising:
a casing having a hollow structure of a predetermined size, wherein the casing comprises an ear specular disposed on a front surface, a display including a liquid crystal display disposed on a rear surface, and a manipulating handle on a lower part;
an image-photographing part comprising a charge-coupled device camera inside the casing, wherein the image-photographing part photographs an ear drum image of a patient through the ear specular; and
a section-photographing part comprising a collimator and a galvanometer mirror inside the casing, wherein the section-photographing part photographs section images of the ear drum and a middle ear of the patient,
wherein the ear drum image obtained by the image-photographing part and the section images of the ear drum and the middle ear obtained by the section-photographing part are displayed in real time on the liquid crystal display such that desirable images can be stored.

2. The diagnosis-and-display integrated optical tomographic imaging otoscope according to claim 1, wherein the casing comprises an illumination visible light source which is disposed on the ear specular to illuminate an inside of an ear of the patient.

3. The diagnosis-and-display integrated optical tomographic imaging otoscope according to claim 1, wherein the image-photographing part comprises magnification lenses downstream of the charge-coupled device camera, a color correction filter mirror downstream of the magnification lenses, and a focus lens downstream of the color correction filter mirror, whereby the charge-coupled device camera photographs the ear drum image of the patient in real time through the ear specular.

4. The diagnosis-and-display integrated optical tomographic imaging otoscope according to claim 3, wherein the color correction filter mirror is disposed at an inclined position, and directs light from the focus lens to the charge-coupled device camera of the image-photographing part by allowing the light to pass through and light from the collimator of the section-photographing part to the focus lens by reflecting the light.

5. The diagnosis-and-display integrated optical tomographic imaging otoscope according to claim 1, wherein the section-photographing part comprises a galvanometer mirror downstream of the collimator, a color correction filter mirror downstream of the galvanometer mirror, and a focus lens downstream of the color correction filter mirror, whereby the section images of the ear drum and the middle ear of the patient are photographed in real time through the ear specular.

6. The diagnosis-and-display integrated optical tomographic imaging otoscope according to claim 5, wherein the color correction filter mirror is disposed at an inclined position, and directs light from the focus lens to the charge-coupled device camera of the image-photographing part by allowing the light to pass through and light from the collimator of the section-photographing part to the focus lens by reflecting the light.

7. The diagnosis-and-display integrated optical tomographic imaging otoscope according to claim 1, wherein the display part concurrently displays the ear drum image obtained from the image-photographing part on one portion of the liquid crystal display and the section images of the ear drum and the middle ear obtained from the section-photographing part on the other portion of the liquid crystal display.

8. The diagnosis-and-display integrated optical tomographic imaging otoscope according to claim 1, wherein the handle has a button on a portion thereof, wherein, when the button is pressed, the ear drum image obtained from the image-photographing part and the section images of the ear drum and the middle ear obtained from the section-photographing part are stored in a memory which is disposed inside a control part.
